(19) United States
(12) Patent Application Publication
(10) Pub. No.: US 2006/0182698 A1
(43) Pub. Date: Aug. 17, 2006

(54) Preparation of chromium oxide pigment with reduced chromium (vi) content

(76) Inventors: Serge Grizzo, Beauchamp (FR); Martine Seu-Salerno, Neuilly sur Seine (FR); Emilie Desmarthon, Magny en Vexin (FR)

Correspondence Address: MICHAEL BEST & FRIEDRICH, LLP, 100 E WISCONSIN AVENUE, MILWAUKEE, WI, 53202, US

(21) Appl. No.: 10/527,949

(22) PCT Filed: Sep. 18, 2003
(86) PCT No.: PCT/FR03/02753

§ 371 Date: Nov. 14, 2005

(30) Foreign Application Priority Data
Sep. 19, 2002 (FR) 02/11619

(51) Int. Cl. A61K 8/19

(52) U.S. Cl. 424/63

(57) ABSTRACT

The invention relates to a method for preparing pigmentary composition of particles based on hydrated chromium oxide for cosmetic use in which chromium is embodied in the form of chromium (VI) in a quantity equal to or higher than 5 ppm with respect to a total mass of said particles (p). The inventive method comprises a stage (E) which consists in putting in contact (a) the particles (p₀), which are based on hydrated chromium oxide and have a chromium (VI) content ranging from 20 to 1000 ppm of the total mass of said particles (p₀), and (b) an iron compound (II).

This invention relates the preparation of pigmentary compositions with a base of hydrated chromium oxide particles with reduced chromium (VI) content, especially the preparation of compositions for cosmetic use.

Chromium oxide pigments, and in particular hydrated chromium oxide pigments, are widely known and used, particularly in paints. These pigments, often called “chromium greens” (anhydrous chromium oxide) or “emerald green” (hydrated chromium oxide), are particles of chromium oxide with a very pronounced green color.

However, although they have very interesting coloring properties, the chromium oxide green pigments currently known also contain significant amounts of chromium (VI), primarily in the form of chromate ions (CrO₄²⁻ or HCrO₄⁻), for example). In these pigments, the chromium (VI) content is generally greater than 100 ppm of the total mass of the composition, with this chromium (VI) content often falling between 200 and 500 ppm.

Because of the high toxicity of chromium (VI), common chromium oxide pigments are not suitable for use as colorants in cosmetics. One could refer, for example to EEC directive 76/768 and annex IV thereto, regarding the use of colorant materials used in cosmetics, which stipulate that a pigment with a chromium oxide base (color index: 77289) must be free of chromate ions to be usable in cosmetics.

However, inventors have now discovered that by treating the common types of chromium oxide pigments with an iron (II)-based reducing agent,

it is possible to quantitatively reduce the chromium (VI) into chromium (III) and to obtain pigments that have a sufficiently reduced chromium (VI) content to allow their use in cosmetic compositions.

On the basis of these discoveries, one goal of this invention is to provide a process for preparing pigmentary compositions with a hydrated chromium oxide base that are non-toxic and non-irritating that can be used in the cosmetics industry in particular.

Thus an object of the present invention is a process for preparing a pigmentary composition comprising particles (p) with a chromium oxide base, in which the chromium present as chromium (VI) represents a maximum of 5 ppm of the total mass of the particles (p), with said process comprising a stage (E) that consists of bringing into contact:

(a) hydrated chromium oxide-based particles (p₀) with a chromium (VI) content between 20 and 1000 ppm (preferably between 100 and 800 ppm, and advantageously between 200 and 500 ppm) of the total mass of said particles (p₀); and

(b) an iron (II) compound, generally in the form of an aqueous solution.

The particles (p₀) used in the process of the invention can be chosen from most hydrated chromium oxide-based pigments known in the state of the art. However, it is generally preferred that the particles (p₀) used have an average size of between 1 and 20 microns, preferably less than or equal to 10 microns (and advantageously less than or equal to 5 microns), with the typical size of the particles (p₀) falling between 3 and 4 microns. The average size of the primary particles of the particles (p₀) is generally on the order of 100 nm.

The process of the invention consists of reducing the chromium (VI) species present to trace amounts in the particles (p₀) using the iron (II) compound, which acts as the reducing agent. In this context, the chromium (VI) is quantitatively reduced to chromium (III) form and the iron (II) is oxidized to iron (III) (in general the formation of Cr(OH)₃ or mixed hydroxides of chromium (III) and iron (III) is also seen), through which we are able to reduce the chromium (VI) content within the particles to a value that is less than or equal to 5 ppm (most often to less than or equal to 1 ppm, advantageously less than or equal to 0.1 ppm, and in the most preferred case less than or equal to 0.01 ppm) of the total amount of chromium present in the particles. In this context, it is generally preferred that the particles (p₀) have an initial chromium (VI) content less than or equal to 750 ppm, advantageously equal to at most 500 ppm (this initial chromium (VI) content typically falling between 200 and 500 ppm), though the process of the invention allows particles with a chromium (VI) content of up to 1000 ppm to be easily treated.

Without being linked in any way to a particular theory, it can be suggested that the reduction process involved according to the invention involves one or more of the following reactions, among others:
3Fe²⁺+Cr⁶⁺→3Fe³⁺+Cr³⁺
8H⁺+3Fe²⁺+CrO₄²⁻→3Fe³⁺+Cr³⁺+4H₂O(if pH≧6.5)
7H⁺+3Fe²⁺+HCrO₄⁻→3Fe³⁺+Cr³⁺+4H₂O(if pH≦6.5)

To perform the optimal reduction reaction, in the process of the invention we prefer that the molar ratio of iron (II) used to the chromium (VI) initially contained in the particles (p₀) be greater than a theoretical ratio of 3:1. The preferred ratio is between 4:1 and 6:1. Advantageously, the ratio is greater than or equal to 4.5:1, and most often we prefer that the ratio remain less than or equal to 5.5:1. This ratio can typically be on the order of 5:1 (i.e., most often between 4.8:1 and 5.2:1)

Most often, the iron (II) compound used in the preparation process of the invention is an iron salt having reducing properties. It is especially advantageous for the iron (II) compound used to be iron (II) sulfate.

In an especially advantageous embodiment, the stage (E) of the process of the invention consists of dispersing the particles (p₀) in a solution (generally water), preferably in the amount of 150 to 300 g of particles per liter (and advantageously between 200 and 250 g of particles per liter), then adding an aqueous solution of the iron (II) compound (generally iron (II) sulfate) to the dispersion obtained. The concentration of the iron (II) solution that is added adjusts naturally to a ratio of added Fe (II): initial Cr (VI) in the ranges cited above. In the context of this particular variation, it is preferred that the aqueous dispersion of the particles (p₀) has a pH between 5 and 9, preferably between 5 and 6. Likewise, it is preferred that the aqueous solution of iron (II) compounds (generally a solution of iron (II) sulfate) has a pH between 5 and 9, and advantageously between 5 and 6.

In the most general case, we prefer that the reaction of the iron (II) compound on the particles (p₀) occur at a pH between 5 and 9, with this pH preferably remaining lower than or equal to 6. In this context, the inventors have demonstrated that, in a pH range between 5 and 9, the rate of reducing the chromium (VI) species into chromium (III) increases as the pH decreases.

As a general rule, it is preferred that stage (E), bringing the particles (p₀) and the iron (II) compound into contact, be performed in a period of time lasting at least 5 hours, and advantageously at least equal to 6 hours, but interesting results can still be obtained with shorter reaction times.

The pigmentary compositions that can be obtained using the process of this invention comprise particles (p) with a hydrated chromium oxide base in which the chromium present as chromium (VI) represents at most 5 ppm (preferably less than 1 ppm, advantageously less than 0.1 ppm and even more advantageously less than 0.01 ppm) of the total mass of the particles (p) in said composition.

By “pigmentary composition comprising particles (p) with a hydrated chromium oxide base,” in this description we mean any composition comprising particles (p) based on a chromium oxide of the formula (Cr₂O₃, nH₂O), in which n designates a number between 1 and 5 and preferably between 2 and 3.

Thus, a pigmentary composition obtained using the process of the invention can advantageously be made in the form of a powder comprising the particles (p), possibly in combination with other constituents such other pigmentary particles or additives, for example, with this powder preferably being in the form of a powder mainly made up of the particles (p). In the case where compositions of the invention are in the form of a powder, they generally comprise at most 5 ppm of chromium (VI) of the total mass of the composition, and preferably at most 1 ppm, advantageously at most 0.1 ppm, and even more preferentially at most 0.01 ppm of the total mass of the composition. This content can be even lower in spray compositions comprising constituents other than the particles (p).

Pigmentary compositions obtained using the invention can also be made in the form of dispersions comprising the particles (p) in suspension in a solvent (generally water or a water-alcohol mixture, if appropriate). If the composition of the invention is in the form of a dispersion in a solvent medium, the dry extract of these compositions (i.e. the solid obtained after) evaporating or eliminating the solvent medium) most often contains at most 5 ppm of chromium (VI) of the total mass of said dry extract (and preferably an amount less than or equal to 1 ppm, advantageously less than or equal to 0.1 ppm, and even more preferentially less than or equal to 0.01 ppm).

Generally, compositions obtained using the invention are suitable for use in cosmetics in the sense of EEC directive 76/768 (Annex IV).

The chromium-oxide based particles (p) in compositions obtained using the invention preferably comprise at least 75 weight %, generally between 75 and 80 weight %, of chromium oxide Cr₂O₃. The particles (p) characteristically comprise water, generally at least 15 weight % (and most often between 15 and 18 weight %).

Further, the particles (p) in a composition obtained using the invention generally have an average size between 0.05 μm and 10 μm. Particles of the invention preferably have a size greater than or equal to 0.5 μm, and advantageously at least equal to 0.8 μm. Most often it is preferred that this size remains less than or equal to 5 μm, and preferably less than or equal to 3 μm. Typically, the particles (p) can have an average size between 1 and 2 μm.

In addition, the particles (p) in a composition using the invention generally have a specific surface area between 90 and 150 m²/g, with this specific surface area preferably equal to at least 100 m²/g, and advantageously at least equal to 110 m²/g. This specific surface area is typically between 110 and 120 m²/g.

In particular, because of their low chromium (VI) content, pigmentary compositions obtained using the process of the invention can especially be used for preparing cosmetic formulations. Thus, as non-limiting manner examples, these compositions can be used in preparing highlighters, mascaras, eyeliners, or for foundation compositions.

The various characteristics and advantages of this invention will become clearer in the illustrative example given below.

### EXAMPLE

A pigmentary composition made up of hydrated chromium oxide particles, in which the average size of the primary particles is 100 nm, comprising 75 weight % chromium oxide and 150 ppm chromium (VI) was treated using the process of the present invention.

For this, a dispersion of 250 g of the pigmentary composition defined above was dispersed in one liter of water. This dispersion was stirred, and the dispersion obtained was acidified by adding a weak acid to obtain a pH of 6. We also made a solution of iron (II) sulfate in a 10 g/L concentration.

We added 100 mL of the iron sulfate solution obtained all at once and left the mixture stirring for 5 hours.

Following this treatment, we filtered the medium obtained and washed the filtrate several times with 1 liter of water until all of the salts obtained were eliminated. The solid obtained was dried and then ground.

At the end of these various treatments, we obtained a pigmentary composition made up of particles with an average primary particle size of 100 mm, characterized by a chromium (VI) content less than 2 ppm of the total mass of the dry composition obtained.

1. A process for preparing a pigmentary composition comprising particles (p) with a chromium oxide base, in which the chromium present as chromium (VI) represents at most 5 ppm of the total mass of the particles (p), said process comprising a stage (E) consisting of bringing into contact:
(a) hydrated chromium oxide-based particles (p₀), with a chromium (VI) content between 20 and 1000 ppm of the total mass of said particles (p₀); and
(b) an iron (II) compound.

2. A process as in claim 1, in which the particles (p₀) used in the process have an average size between 1 and 20 microns.

3. A process as in claim 1, in which the molar ratio of the iron (II) used to the chromium (VI) initially found in the particles (p₀) is between 4:1 and 6:1.

4. A process as in claim 1, in which the iron (II) compound is iron (II) sulfate.

5. A process as claim 1, in which the reaction of the iron (II) compound used has a pH between 5 and 9.

6. A Process as in claim 1 for the preparation of a pigmentary composition intended for use in a cosmetic formulation.
